(19) United States
(12) Patent Application Publication
(10) Pub. No.: US 2016/0136202 A1
(43) Pub. Date: May 19, 2016

(54) ANTI-VIRAL WASH

(71) Applicant: Travis Honeycutt, Gainesville, GA (US)

(72) Inventor: Travis Honeycutt, Gainesville, GA (US)

(21) Appl. No.: 14/939,337

(22) Filed: Nov. 12, 2015

Related U.S. Application Data
(60) Provisional application No. 62/079,383, filed on Nov. 13, 2014.

(51) Int. Cl. A61K 33/42; A61K 33/00; A61K 33/08

(52) U.S. Cl. CPC A61K 33/42; A61K 33/08; A61K 33/00

(57) ABSTRACT

An anti-viral wash composition composed of approximately 0.005% to 1.0% (wt.) aluminum hydroxide, approximately 0.1% to 0.001% (wt) sodium bicarbonate and approximately 0.1% to 0.001% (wt) trisodium phosphate.

### RELATED APPLICATIONS

The present application relies upon provisional patent application No. 62/079,383, filed on Nov. 13, 2014.

### TECHNICAL FIELD

The present invention is to a topical application to incapacitate virus contamination acquired during contact with infected patients and victims. This product uses only chemical compounds regarded as Generally Recognized as Safe (GRAS) ingredients by the FDA.

### BACKGROUND OF THE INVENTION

Viruses have different mechanisms by which they produce disease in an organism, depending largely on the viral species. Mechanisms at the cellular level primarily include cell lysis, the breaking open and subsequent death of the cell. In multicellular organisms, if enough cells die, the whole organism will start to suffer the effects. Although viruses cause disruption of healthy homeostasis, resulting in disease, they may exist relatively harmlessly within an organism. Most people have been infected with at least one latent virus, such as the herpes virus which, can be beneficial as the presence of the virus can increase immunity against bacterial pathogens. Some viruses can cause lifelong or chronic infections, where viruses continue to replicate in the body despite the host defensive mechanisms. This is common in the hepatitis B virus and hepatitis C virus infections. People chronically infected are known as carriers, as they serve as reservoirs of infectious virus. In populations with high proportion of carriers, diseases said to be endemic.

Horizontal transmission is the most common mechanism of the spread of viruses in populations. Transmission can occur when bodily fluids are exchanged during sexual activity, for example, the HIV virus or when blood is exchanged by contaminated transfusion or needle sharing, common in the spread of the hepatitis C virus or an exchange of saliva by mouth, for example, the Epstein-bar virus or when contaminated food or water is ingested, for example in the spread of the neuro-virus.

As epidemiology is used to break the chain of infection in populations during outbreaks of viral diseases, it is thought that a simple wash could be developed to reduce the spread of a number of viruses. This approach would be particularly advantageous as an adjunct to vaccinations that provide immunity to infection and antiviral drugs that selectively interfere with viral replication recognizing that viruses use vital metabolic pathways within host cells to replicate and are thus difficult to eliminate without using drugs because toxic of their affects to host cells in general.

### SUMMARY OF THE INVENTION

An anti-viral wash comprising approximately 0.005% to 1.0% (wt)) aluminum hydroxide, approximately 0.1% to 0.001% (wt.) sodium bicarbonate and approximately 0.1% to 0.001% (wt) trisodium phosphate.

### DETAILED DESCRIPTION OF THE INVENTION

Novel features which are characteristic of the invention, as to organization and method of operation, together with further objects and advantages thereof will be better understood from the following description. The various features of novelty which characterize the invention are recited with particularity in the claims.

There have been broadly outlined important features of the invention in the summary presented above. There are, of course, additional features of the invention that will be described hereinafter and which will form additional subject matter of the claims appended hereto. Those skilled in the art will appreciate that the conception upon which this disclosure is based readily may be utilized as a basis for the designing of other structures, methods and systems for carrying out the several purposes of the present invention. It is important therefore, that claims be regarded as including such equivalent constructions insofar as they do not depart from the spirit and scope of the present invention.

The present invention is a topical application to incapacitate virus contamination acquired during contact with infected patients and victims. This product uses only chemical compounds regarded as Generally Recognized as Safe (GRAS) ingredients by the FDA.

The active ingredients are aluminum hydroxide (AL(OH)₃) and sodium bicarbonate (NaHCO₃). Briefly, aluminum hydroxide has the capacity to bind or immobilize up to nine sites or electrons on protein backbones (in the virus skeleton) through electron pairing, electron sharing, free radical accommodation, charge neutralization or Van der Waal charge sharing. These sites are found on proteins on nitrogen atoms (2s² electrons) and sulfur, as well as other elements. These sites are found in abundance on protein rich viruses. Sodium bicarbonate is a widely used compound used as a buffer and wetting agent in a variety of medicines, cosmetics, soaps, and food. Together in sufficient concentration these compounds are capable of incapacitating all Ebola virus contacting the product.

It is anticipated that this invention eventually will be the universally accepted rinse product used by hospitals, emergency centers, and healthcare workers threatened by exposure to protein rich viruses, such as influenza, polio, AIDS, and Ebola. It is non-toxic and safe for the purposes described herein. In fact, it is recommended that it be used to rinse the eyes and all mucous membranes subject to possible contamination. Typically, an Anti-Viral Wash rinse would be followed by commonly applied antibacterial disinfectants in accordance with current institutional protocols. Any subsequent disinfectant protocols will not compromise the anti-viral effects of its initial application.

The product's applications may be packaged and dispensed as liquid topical sprays, drops or debridement applicators; as gel saturated swabs or sponges; or water dispensers treated, high volume bulk dispenser for full body rinsing. Possibly, the product may have internal applications. The product manufacturing is straightforward using mixing and production techniques widely used currently.
0.005% to 1.0% Aluminum Hydroxide is sufficient and 0.5% works but 0.1% is satisfactory
0.1% to 0.001% Sodium Bicarbonate was workable but 0.05% was best
0.1% to 0.001% Trisodium Phosphate was workable but 0.05% was best

This use was surprising as aluminum hydroxide is used in water treatment as a filtering agent and in thin layer chromatography as a solid to immobilize impurities. Here the solid is dispersed in a liquid to capture protein rich virus.

The above disclosure is sufficient to enable one of ordinary skill in the art to practice the invention, and provides the best mode of practicing the invention presently contemplated by the inventor. While there is provided herein a full and complete disclosure of the preferred embodiments of the invention, it is not desired to limit the invention to the exact construction, dimensions, relationships, or operations as described. Various modifications, alternative constructions, changes and equivalents will readily occur to those skilled in the art and may be employed as suitable without departing from the true spirit and scope of the invention. Such changes might involve alternative materials, components, structural arrangements, sizes, shapes, forms, functions, operational features or the like. Therefore, the above description and illustration should not be considered as limiting the scope of the invention, which is defined by the appended claims.

What is claimed is:

1. An anti-viral wash composition comprising approximately 0.005% to 1.0% (wt.) aluminum hydroxide, approximately 0.1% to 0.001% (wt.) sodium bicarbonate and approximately 0.1% to 0.001% (wt.) trisodium phosphate.

2. The anti-viral wash composition of claim 1 wherein said composition comprises approximately 0.1% to 0.5% (wt.) aluminum hydroxide.

3. The anti-viral wash composition of claim 1 wherein said composition comprises approximately 0.05% (wt.) sodium bicarbonate.

4. The anti-viral wash composition of claim 1 wherein said composition comprises approximately 0.05% trisodium phosphate.
